SEQUENCE LISTING (10 sequences)

SEQ ID NO: 1 - DNA, 23 bp, Artificial
  primer
gggatggaattgtgagggagatg

SEQ ID NO: 2 - DNA, 24 bp, Artificial
  primer
tgatgctggtgctgagtatgtcgt

SEQ ID NO: 3 - DNA, 20 bp, Artificial
  primer
tgccaactcagtgaggacaa

SEQ ID NO: 4 - DNA, 20 bp, Artificial
  primer
tccaacaggcctgagaaatc

SEQ ID NO: 5 - DNA, 22 bp, Artificial
  primer
tccacacggaatgctgccatgg

SEQ ID NO: 6 - DNA, 22 bp, Artificial
  primer
agcggcacagcacttctctaga

SEQ ID NO: 7 - DNA, 25 bp, Artificial
  primer
gacattcctcagtgttcagaagttg

SEQ ID NO: 8 - DNA, 21 bp, Artificial
  primer
tgcctgattctgtgtgatcca

SEQ ID NO: 9 - DNA, 20 bp, Artificial
  primer
tggtatcgtggaaggactca

SEQ ID NO: 10 - DNA, 20 bp, Artificial
  primer
agtgggtgtcgctgttgaag

(19) United States
(12) Patent Application Publication
(10) Pub. No.: US 2009/0232781 A1
(43) Pub. Date: Sep. 17, 2009

(54) Treatment of liver diseases through transplantation of human umbilical mesenchymal stem cells

(75) Inventor: Yu-Show Fu, Taipei (TW)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignees: Yu-Show Fu, Taipei (TW); Henrich Cheng, Taipei (TW)

(21) Appl. No.: 12/378,254

(22) Filed: Feb. 12, 2009

Related U.S. Application Data
(60) Provisional application No. 61/069,326, filed on Mar. 13, 2008.

(51) Int. Cl. A61K 35/12; A61P 1/16

(52) U.S. Cl. 424/93.7

(57) ABSTRACT

A method for treating liver diseases or liver damage, including but not limited to liver fibrosis, and/or aiding recovery from liver diseases, including but not limited to liver fibrosis, or liver damage in a subject, includes transplanting human umbilical mesenchymal stem cells (HUMSCs) obtained from Wharton's Jelly to the area of liver disease or damage of the subject.

### CROSS-REFERENCE TO RELATED APPLICATION

This application claims the benefit under 35 U.S.C. §119(e) of U.S. Provisional Patent Application No. 61/069,326, filed Mar. 13, 2008, the entire disclosure of which is hereby incorporated by reference herein.

### BACKGROUND OF THE INVENTION

Fu et al. developed and reported human umbilical mesenchymal stem cells (HUMSCs) and their properties, which are the stem cells from the mesenchymal tissue from the Wharton's jelly of the umbilical cord (Fu, Y. S. et al., Conversion of Human Umbilical Cord Mesenchymal Stem Cells in Wharton's Jelly to Dopaminergic Neurons in Vitro—Potential Therapeutic Application for Parkinsonism. Stem Cells 2004; 24: 115-124). It was reported that human umbilical cord mesenchymal stem cells (HUMSCs) could be induced to differentiate into neuron-like cells (about 87%), expressing neurofilament, functional mRNAs responsible for the syntheses of subunits of the kainate receptor and glutamate decarboxylase and generating an inward current in response to evocation by glutamate (Fu, Y. S. et al., Transformation of Human Umbilical Mesenchymal Cells into Neurons in Vitro. Journal of Biomedical Science 2004; 11: 652-660). It was also reported that HUMSCs were capable of differentiating into osteogenic, chondrogenic, adipogenic, and myogenic in vitro (Wang, H. S., Hung, S. C., and Pong, S. T., Mesenchymal Stem Cells in Wharton Jelly of the Human Umbilical Cord. Stem Cells 2004; 22: 1330-1337).

Chronic injury leading to liver fibrosis occurs in response to a variety of factors, including viral hepatitis, alcohol abuse, drugs, metabolic diseases, autoimmune attack of hepatocytes or bile duct epithelium, or congenital abnormalities. Typically, injury is present for months to years before significant scar accumulates, although the time course may be accelerated in congenital liver disease. Liver fibrosis is reversible, whereas cirrhosis, the end-stage consequence of fibrosis, is generally irreversible. Thus, efforts to understand fibrosis focus primarily on events that lead to the early accumulation of scar in the hope of identifying therapeutic targets to slow its progression.

Liver fibrosis is characterized by excessive accumulation of extracellular matrix, with the formation of scar tissue encapsulating the area of injury. Liver fibrosis results in many clinical manifestations, including ascites, variceal hemorrhage, and encephalopathy. The prognosis for patients with the disease is poor, although liver transplantation is a good alternative treatment. However, there are limited available donor livers for millions of patients worldwide. Therefore, exogenous cell replacement strategies have to be considered. The present invention is an effective treatment for liver disease involving a novel exogenous cell replacement treatment.

### BRIEF SUMMARY OF THE INVENTION

The present invention is based on the discovery of the therapeutic effects of transplantation of HUMSCs, particularly with respect to treatment of liver disease.

Accordingly, one aspect of the present invention relates to a method for treating a liver disease or aiding recovery from a liver disease in a subject comprising transplanting human umbilical mesenchymal stem cells (HUMSCs) obtained from Wharton's Jelly to areas of the liver disease of the subject. The liver disease is selected from the group consisting of liver inflammation, liver steatosis, liver fibrosis, liver cirrhosis, and hepatitis.

Another aspect of the present invention also relates to a method for enhancing liver regeneration and inhibiting liver inflammation in a subject with liver damage comprising transplanting HUMSCs obtained from Wharton's Jelly to the areas of liver damage of the subject.

The details of one or more embodiments of the invention are set forth in the description below. Other aspects or advantages of the present invention will be apparent from the following detailed description of several embodiments, and also from the appended claims.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the embodiments shown in the drawings.

In the drawings:

FIG. 1 comprises FIGS. 1A-1C. FIG. 1A is a graph showing the weight changes of the rats in all groups before feeding with CCl₄ and after feeding with CCl₄ for 8 weeks (W). FIG. 1B is a graph showing the serum glutamyl pyruvic transaminase (GPT) levels obtained and detected from the blood of the rats in all groups. FIG. 1C is a graph showing the serum glutamyl oxaloacetic transaminase (GOT) levels obtained and detected from the blood of the rats in all groups. The results showed that the serum GPT and GOT levels of the rats fed with CCl₄ for 4 or 8 weeks increased significantly compared to the normal group which was not fed with CCl₄. * indicates significant difference at p<0.05 between the CCl₄ (8 W), CCl₄ (8 W)+HUMSCs (liver), CCl₄ (8 W)+HUMSCs (tail v) groups compared with the normal group at the same time point. # means there was a distinct difference between the results at 8-weeks and 4-weeks in the same group (paired t-test, p<0.05).

FIG. 2 comprises FIGS. 2A-2K. FIGS. 2A-2D are photographs of fresh livers without fixation after the 8-week experiment. The results showed that the livers of the normal group were smooth, lustrous, and reddish (FIG. 2A). As for the rats fed with CCl₄ for 4 weeks (CCl₄ (4 W)), the liver surfaces were coarse and relatively bloodless (FIG. 2B). The liver surfaces of the rats fed with CCl₄ for 8 weeks were coarser, with nodules found, and nearly bloodless (FIG. 2C). For the livers of the CCl₄ (8 W)+HUMSCs (liver) group, the liver surfaces were slightly coarse, but were more reddish and lustrous (FIG. 2D) compared to those of the CCl₄ (4 W) and CCl₄ (8 W) groups.

FIGS. 2E-2I are photomicrographs showing Sirius red staining of liver sections from the following experimental groups: normal rats (FIG. 2E); CCl₄ (4 W) (FIG. 2F); CCl₄ (8 W) (FIG. 2G); CCl₄ (8 W)+HUMSCs (tail v.) (FIG. 2H); and CCl₄ (8 W) +HUMSCs (liver) (FIG. 2I). The scale bar in FIG. 2I, also regarding FIGS. 2E-2H, represents a distance of 100 μm.

FIG. 2J is a graph showing the quantification of the percentage of liver fibrosis labeled by Sirius red staining in rat livers. The results showed that the liver fibrosis (reddish areas) of the rats of the CCl₄ (4 W), CCl₄ (8 W), and CCl₄ (8 W)+HUMSCs (tail v.) groups was very obvious.

FIG. 2K is a graph showing the quantification of the collagen concentration in fresh rat livers by the soluble collagen assay kit. The results showed that there was no statistical difference between the collagen levels of the CCl₄ (8 W)+HUMSCs (liver) group and the normal group. * indicates a significant difference at p<0.05 between the CCl₄ (4 W), CCl₄ (8 W), CCl₄ (8 W)+HUMSCs (tail v) groups compared with the normal group at the same time point. # means there was a significant difference between the CCl₄ (8 W) and CCl₄ (8 W)+HUMSCs (tail v) groups compared with the CCl₄ (4 W) group (p<0.05).

FIG. 3 comprises FIGS. 3A-3D. FIG. 3A includes a photograph of a western blot and a graph showing the results of western blotting of α-smooth muscle actin (α-SMA) level in the livers. FIG. 3B is a graph showing the transforming growth factor (TGF) β-1 protein level detected by ELISA. FIG. 3C includes a photograph of a western blot and a graph showing the results of western blotting of Met-P level in the livers. FIG. 3D is a graph showing the mRNA level of hepatocyte growth factor (HGF) in the grafted liver of the rat by real time reverse transcriptase polymerase chain reaction (RT-PCR). * indicates a significant difference at p<0.05 vs. the normal group. # means there was a significant difference between the CCl₄ (8 W) group compared with the CCl₄ (8 W)+HUMSCs (liver) group (p<0.05).

FIG. 4 comprises FIGS. 4A-4L. FIGS. 4A-4H are photomicrographs showing the existence and distribution of HUMSCs in rat livers 4 weeks after transplantation; identification of anti-human specific nuclear antigen positive cell bodies in the grafted liver of the CCl₄ (8 W)+HUMSCs (tail v) group (FIGS. 4A-4D) and the CCl₄ (8 W)+HUMSCs (liver) group (FIGS. 4E-4H). FIGS. 4B, 4D, 4F, 4G and 4H are magnified images of the boxed areas in FIGS. 4A, 4C and 4E, respectively. The scale bar in FIG. 4H, regarding FIGS. 4B, 4D, 4F, 4G and 4H represents a distance of 100 μm.

FIGS. 4I-4K are photomicrographs showing double fluorescence immunohistochemistry of human α-fetoprotein and albumin proteins in the livers of the CCl₄ (8 W)+HUMSCs (liver) group. The anti-human specific nuclear antigen positive cell bodies (red, arrows in FIG. 4I) were human albumin negative cytoplasma (green in FIG. 4J) in grafted livers of the CCl₄ (8 W)+HUMSCs (liver) group. FIG. 4K is the merged result of FIGS. 4I and 4J. The scale bar in FIG. 4K, regarding FIGS. 4I and 4J represents a distance of 20 μm.

FIG. 4L is a photograph showing the results of RT-PCR of human α-fetoprotein and albumin proteins in the livers of the CCl₄ (8 W)+HUMSCs (liver) group. Lane 1: positive control (human hepatoma liver); Lane 2: liver tissue from normal rats; Lane 3: liver tissue from the CCl₄ (8 W) group; Lane 4: liver tissue from the CCl₄ (8 W)+HUMSCs (liver) group.

FIG. 5 comprises FIGS. 5A-5G and shows the results of analysis by human cytokine antibody array of grafted livers of the CCl₄ (8 W)+HUMSCs (liver) group. The intensities of the relative expression levels of cytokines were quantified by densitometry. The liver samples are from normal rats (FIGS. 5A and 5D), from the CCl₄ (8 W) group (FIGS. 5B and 5E) and from the CCl₄ (8 W)+HUMSCs (liver) group (FIGS. 5C and 5F), respectively. FIG. 5G is a graph showing the relative density of cutaneous T cell-attracting chemokine (CTACK), leukemia inhibitory factor (LIF) and prolatin in the livers of the normal group, the CCl₄ (8 W) group and the CCl₄ (8 W)+HUMSCs (liver) group. * indicates a significant difference at p<0.05 vs. the normal group.

FIG. 6 comprises FIGS. 6A-6I, and are photographs using microPET™ positron emission tomography imaging for small animals demonstrating transplantation of HUMSCs into fibrosis livers. The rats were injected with ¹⁸F-fluorodeoxyglucose (FDG) and microPET™ was used to scan the rats' abdomens to verify if there was hepatitis or hepatoma. The metabolic rate of the livers was very low before the rats were fed with CCl₄ (FIGS. 6A-6C). After feeding with CCl₄ for 4 weeks, the metabolic rate of the rat livers increased significantly (FIGS. 6D-6F). Four weeks after HUMSC transplantation, the metabolic rate of the rat livers dropped to the level before treatment (FIGS. 6G-6I). FIGS. 6A, 6D, 6G are horizontal sections; FIGS. 6B, 6E, 6H are coronal sections; and FIGS. 6C, 6F, 6I are saggital sections.

### DETAILED DESCRIPTION OF THE INVENTION

As used herein the following terms may be used for better interpretation of claims and the specification.

The articles “a” and “an” are used herein to refer to one or more than one (i.e., at least one) of the grammatical object of the article. By way of example, “an element” means one element or more than one element.

As used herein, the term “human umbilical mesenchymal stem cell (HUMSC)” refers to cells of the mesenchymal tissue in human umbilical cord (i.e., the so-called Wharton's Jelly).

As used herein, the term “liver fibrosis” refers to the accumulation of interstitial or “scar” extracellular matrix after either acute or chronic liver injury. Cirrhosis, the end-stage of progressive fibrosis, is characterized by septum formation and rings of scar that surround nodules of hepatocytes. Typically, fibrosis requires years or decades to become clinically apparent, but notable exceptions in which cirrhosis develops over months may include pediatric liver disease (e.g. biliary atresia), drug-induced liver disease, and viral hepatitis associated with immunosuppression after liver transplantation.

As used herein, the phrase “recovery from liver fibrosis” refers to improvement in the pathological conditions of the subject suffering from liver fibrosis and/or restoration (at least partially) of the physical function of the injured liver. For example, in the embodiment used in the examples of the present invention, the liver surface of HUMSCs transplantation were more reddish and lustrous compared to those of the CCl₄ (4 W) and CCl₄ (8 W) group. In another embodiment used in the examples of the present invention, the amount of collagen was less in the groups of HUMSCs transplantation. In such case, recovery from the liver fibrosis means regeneration of the liver cells and reduction of collagen in the liver.

As used herein, a “subject” includes human and non-human animals having a liver.

The present invention relates to the use of HUMSCs obtained from Wharton's Jelly in treating a liver disease and/or aiding recovery from a liver disease. Accordingly, the present invention relates to a method for treating a liver disease and/or aiding recovery from a liver disease in a subject comprising transplanting the HUMSCs obtained from Wharton's Jelly to the area of the liver disease of the subject. The liver disease is selected from the group consisting of liver inflammation, liver steatosis, liver fibrosis, liver cirrhosis, and hepatitis. In one embodiment, HUMSCs obtained from Wharton's Jelly was used to treat liver fibrosis.

To transplant HUMSCs to the area of liver disease, the HUMSCs may be administered systemically via a parenteral route, or directly delivered to the liver, such as by means of injection. The term “parenteral” as used herein refers to subcutaneous, intracutaneous, intravenous, intramuscular, intra-arterial, intralesional, as well as any suitable infusion technique, The HUMSCs may be delivered to the area of liver disease neat or as part of a composition with a pharmaceutically acceptable vehicle which may be, for example, mannitol, water, Ringer's solution, and isotonic sodium chloride solution. In a preferred embodiment of the present invention, the HUMSCs are delivered to the desired area in direct liver injection or intravenous injection or infusion.

The amount of HUMSCs to be transplanted to the area of the liver disease varies in view of many parameters, such as the conditions of the subject and the type and severity of the liver disease. The amount of HUMSCs, when applied to the subject suffering from liver disease, should attain a desired effect, i.e., repair the liver disease and/or enhance at least partially functional recovery of the injured liver disease. A suitable amount can be readily determined in view of the present disclosure by persons of ordinary skill in the art without undue experimentation. In a preferred embodiment of the present invention, the amount of HUMSCs transplanted to the area of the liver disease is about 10⁴ to about 10⁷ cells.

The method of the present invention can also enhance liver regeneration and/or inhibiting liver inflammation with liver damage in a subject. Liver damage from any source may result in liver regeneration, necrosis (cell death), degeneration, inflammation, fibrosis, or combinations of these processes. With progressive injury, disruption of liver function can have life-threatening consequences. Cirrhosis, which is a common end-stage liver disease, is one of the top ten causes of death in the Western world. In one embodiment, the present invention used a model to induce fibrosis, which was the source of inflammation. With the treatment of HUMSCs delivered to the desired area in direct liver injection or intravenous injection, liver regenerated from the damage substantially. Moreover, the inflammation in the area of liver damage was reduced. Such therapeutic effects are demonstrated in the following examples.

Without further elaboration, it is believed that one skilled in the art can, based on the above description, utilize the present invention to its fullest extent. The following specific examples relate to that are, therefore, to be construed as merely illustrative, and not limitative of the present invention in any way whatsoever.

### EXAMPLES

Materials and Methods

Preparation of HUMSCs from Wharton's Jelly

Human umbilical cords were collected in Hank's Balanced Salt Solution (HBSS; Gibco® 14185-052, USA) at 4° C. Following disinfection in 75% ethanol for 30 s, the umbilical cord vessels were cleared off while still in HBSS. The mesenchymal tissue (in Wharton's jelly) was then diced into cubes of about 0.5 cm³ and centrifuged at 250 g for 5 min. Following removal of the supernatant fraction, the precipitate (mesenchymal tissue) was washed with serum-free DMEM (Gibco® 12100-046, Invitrogen Corp.) and centrifuged at 250 g for 5 min. Following aspiration of the supernatant fraction, the precipitate (mesenchymal tissue) was treated with collagenase at 37° C. for 18 h, washed, and further digested with 2.5% trypsin (Gibco® 15090-046, Invitrogen Corp.) at 37° C. for 30 min. Fetal bovine serum (FBS; HyClone® SH30071.03, Thermo Fisher Scientific Inc.) was then added to the mesenchymal tissue to neutralize the excess trypsin. The dissociated mesenchymal cells were further dispersed by treatment with 10% FBS-Dulbecco's Modified Eagle Medium (DMEM) and counted under a microscope with the aid of a hemocytometer. The mesenchymal cells were then used directly for cultures or stored in liquid nitrogen for later use.

Animal Grouping

For the different treatment combinations, rats were divided into five experimental groups: (1) normal group: the rats received the same volume of paraffin oil alone; (2) CCl₄ (4 W) group: the rats were treated with CCl₄ for 4 weeks; (3) CCl₄ (8 W) group: the rats were treated with CCl₄ for 8 weeks; (4) CCl₄ (8 W)+HUMSCs (tail vein) group: the rats were treated with CC14 for 8 weeks; one day after the eighth treatment of CCl₄, the rats received 10⁶ HUMSCs via tail vein injection; (5) CCl₄ (8 W)+HUMSCs (liver) group: the rats were treated with CCl₄ for 8 weeks; one day after the eighth treatment of CCl₄, the rats received 5×10⁵ HUMSCs injected into the right liver.

Animal Model of CCl₄-Induced Liver Fibrosis

Male Sprague-Dawley rats (250-300 g body weight, obtained from the Animal Center of National Yang-Ming University, Taiwan) were bred and maintained in an air-conditioned animal house with specific pathogen-free conditions, and were subjected to a 12:12-h daylight/darkness condition and allowed unlimited access to chow and water. Rats were treated with a mixture of CCl₄ and olive oil (1:1 (v/v); at a dose of 0.5 ml/kg body weight of mixture the first time and 1 ml/kg for the remaining time) by gavage tube twice a week for 8 weeks. The rats were sacrificed at 1 day after the eighth or sixteenth CCl₄ treatment. After harvesting the livers, left lobes were removed for histological examination and western blotting, and the right livers were freshly frozen for soluble collagen assay, human cytokine array assay, RT-PCR and protein extraction.

Examination of Serum Level of Glutamyl Oxaloacetic Transaminase (GOT) and Glutamyl Pyruvic Transaminase (GPT)

In order to assay the function of the liver, GOT and GPT in serum were assessed using Binesion™ equipment in the Department of Medical Technology, Taipei Veterans General Hospital, Taiwan.

Quantitative Analysis of Liver Fibrosis From Liver Sections

Ten μm liver sections were fixed with 4% paraformaldehyde in 0.1 M phosphate buffer for 20 min and then washed with 0.1 M phosphate buffer. Tissue sections were stained in 0.1% sirius red in picric acid solution followed by dehydration and mounting with permount. The liver fibrosis area was quantified with sirius red staining using an Olympus microscope equipped with a CCD camera. The red area, considered the fibrotic area, was assessed by computer-assisted image analysis with Image-Pro® software. The mean value of six randomly selected areas per section from six left liver sections in each rat was used as the expressed percent area of fibrosis.

Double Staining of Anti-Human-Specific Nuclear Antigen and Anti-Human Albumin

For the assessment of the possible differentiation of HUMSCs into hepotocytes, double staining was applied for human-specific nuclear antigen (Zhang, S. C. et al., In vitro differentiation of transplantable neural precursors from human embryonic stem cells. Nature Bio. 2001, 19:1129-1133) and human albumin. In order to trace the survival of HUMSCs, anti-human specific nuclear antigen immunostaining was performed. Ten μm liver sections were fixed with 4% paraformaldehyde in 0.1 M phosphate buffer for 20 min and then washed with 0.1 M phosphate buffer. They were then treated with a blocking solution for 30 min in order to prevent nonspecific antibody-antigen binding. The liver sections were then reacted with primary antibodies (mouse anti-human-specific nuclear antigen, 1:25, Chemicon International, Inc. MAB1281, 1:100; mouse anti-human albumin antibody, Sigma A6684, 1:100) at 4° C. for 18 h, washed with 0.1 M phosphate buffered saline (PBS), reacted with secondary antibodies (Fluorescein-conjugated goat anti-mouse-IgG for human nuclei, 1:50, Chemicon International, Inc. AP124F; Rhodamine-conjugated-goat anti-mouse-IgG for albumin, 1:50, Chemicon AP124R) at room temperature for 1 h. The liver sections were then observed under a fluorescence microscope.

Protein Extraction and Western Blotting

Liver tissues were rinsed twice with PBS (4° C.) and lysed with lysis buffer containing 150 mmol/L NaCl, 1.5 mmol/L MgCl², 5 mmol/L EDTA, 1% Triton® X-100 surfactant, 1% NP40, 10 mmol/L NaF, 1 mmol/L Na₃VO₄, and protease inhibitor cocktail (Roche, Indianapolis, Ind.). The protein concentration of the tissue homogenates was determined by the BCA protein assay kit (Pierce Biotechnology, Rockford, Ill.). SDS-PAGE (10% acrylamide gel) and Western blotting were performed as previously described.

Quantitative Analysis of Liver Fibrosis From Fresh Liver

This assay employed Sircol™ Soluble Collagen Assay kit (Biodye Science S1000) to determine the amount of liver collagen.

The right liver was taken out and frozen in liquid nitrogen. The 50 mg tissues were homogenized and then centrifuged in 2.5 mg pepsin of 0.5 M acetic acid solution. The supernatant was reacted with the Sircol™ dye reagent, and then centrifuged at 25000 g for 30 min. The resulting pellet was mixed with Sircol™ alkali reagent. The eluted color was read immediately in a spectrophotometer at 540 nm, i.e., the wavelength corresponding to the maximal absorbance of Sirius red.

Reverse Transcription-Polymerase Chain Reaction (RT-PCR) Detection of the Human Albumin and α-fetoprotein in Transplanted Rat Livers

Total RNA was freshly isolated from rat livers using Tri Reagent™ (Sigma), reverse transcribed into first strand cDNA using oligo(dT) primer, and amplified by 35 cycles (94° C., 1 min; 55° C., 1 min; and 72° C., 1 min) of PCR using 10 pmole of specific primers. On completion of the PCR, products were examined on 2% agarose gel. Rat glyceraldehyde 3-phosphate dehydrogenase (GAPDH) primers were used as an internal standard. Human hepatoma was used as a positive control. Primer Sequences:

Rat GAPDH

(SEQ ID NO: 1)

| | |
|---|---|
| Sense: | 5′-GGGATGGAATTGTGAGGGAGATG-3′ |

(SEQ ID NO: 2)

| | |
|---|---|
| Antisense: | 5′-TGATGCTGGTGCTGAGTATGTCGT-3′ |

Human α-Fetoprotein

(SEQ ID NO: 3)

| | |
|---|---|
| Sense: | 5′-TGCCAACTCAGTGAGGACAA-3′ |

(SEQ ID NO: 4)

| | |
|---|---|
| Antisense: | 5′-TCCAACAGGCCTGAGAAATC-3′ |

Human Albumin

(SEQ ID NO: 5)

| | |
|---|---|
| Sense: | 5′-TCCACACGGAATGCTGCCATGG-3′ |

(SEQ ID NO: 6)

| | |
|---|---|
| Antisense: | 5′-AGCGGCACAGCACTTCTCTAGA-3′ |

Real-Time PCR

Complementary DNA was produced from cellular RNA (5 μg) using a SuperScript® II RNase H-Reverse Transcriptase Kit (Invitrogen, Carlsbad, Calif.). Real-time PCR primers were designed using PRIMER EXPRESS® software (Version 1.5, Applied Biosystems) and the specificity of sequences was verified using BLAST (ncbi.nlm.gov/BLAST/). Reactions were performed in 10-μl quantities of diluted cDNA sample, primers (100, 200, or 300 nM), and a SYBR® Green PCR Master Mix containing nucleotides, AmpliTaq® Gold DNA polymerase, and optimized buffer components (Applied Biosystems). Reactions were assayed using an Applied Biosystems Prism® 7700 sequence detection system. The primers used for real-time PCR were shown as the following:

Rat heptaocyte growth factor (HGF)

(SEQ ID NO: 7)

| | |
|---|---|
| Sense: | 5′-GACATTCCTCAGTGTTCAGAAGTTG-3′ |

(SEQ ID NO: 8)

| | |
|---|---|
| Antisense: | 5′-TGCCTGATTCTGTGTGATCCA-3′ |

Rat GAPDH

(SEQ ID NO: 9)

| | |
|---|---|
| Sense: | 5′-TGGTATCGTGGAAGGACTCA-3′ |

(SEQ ID NO: 10)

| | |
|---|---|
| Antisense: | 5′-AGTGGGTGTCGCTGTTGAAG-3′ |

Human Protein Cytokine Array

In order to elucidate which human cytokines might be involved in the repair of liver fibrosis, a human protein cytokine kit (RayBio® Human Cytokine Antibody Array C Series 2000, RayBiotech, Inc. AAH-CYT-2000) was used for the human protein cytokine assay. The rat liver tissue was homogenized and centrifuged in 1× cell lysis buffer at 1,500 g to remove cell debris. The harvested supernatant was used for the assay of cytokines proteins using a human protein cytokine array kit. The membranes included in the human protein cytokine array kit were blocked with a blocking buffer, and then 1 ml of sample supernatant was individually added and incubated at room temperature for 2 h. The membranes were then analyzed according to the manufacturer's instructions.

### Example 1

### Direct Transplantation of HUMSCs into Rat Livers with Liver Fibrosis Effectively Reduced Serum GOT and GPT Levels

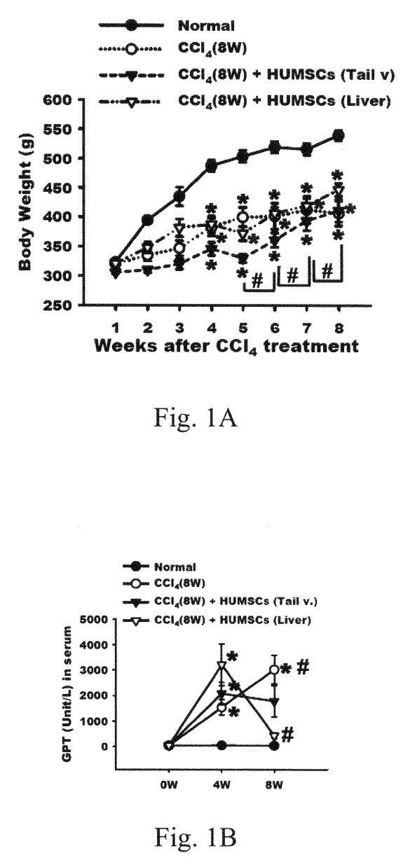

In the 8-week experiment, the weight of the rats which were fed with only olive oil (normal group) increased from 323±4 g to 540±8 g (FIG. 1A). The weight of the rats which were fed with CCl₄ for 4-8 weeks decreased significantly (p<0.05) compared to the normal group. Moreover, some rats had serious ascites. For the rats in the CCl₄ (8 W)+HUMSCs (tail v.) group, a significant drop in body weight was noted, compared to the normal group (p<0.05); no statistical difference (p>0.05, FIG. 1A) was found between the body weight of the rats and that of the CCl₄ (8 W) group. However, in the 5th-8th weeks, the rats of CCl₄ (8 W)+HUMSCs (tail v.) group showed a distinct trend in increased weight (p<0.05, FIG. 1A).

Comparing the body weight of the rats in the CCl₄ (8 W)+HUMSCs (liver) group with the normal group, a significant decrease (p<0.05) was also found; there was no statistical difference (p>0.05, FIG. 1A) between the weight of the rats and that of the CCl₄ (8 W) group and CCl₄ (8 W)+HUMSCs (tail v) group. However, in the 5th-8th weeks, the rats of the CCl₄ (8 W)+HUMSCs (liver) group showed a distinct trend in increased weight (p<0.05, FIG. 1A).

The serum GOT level of the normal group was 121.30±6.93-143.60±5.73 Unit/L (FIG. 1C). After feeding the rats with CCl₄ for 4 weeks, the GOT level increased significantly to 2150.22±388.21 Unit/L (p<0.05). By continuous feeding until the 8th week, the GOT level increased further to 5122.11±998.85 Unit/L (p<0.05) (FIG. 1C). For the rats which received HUMSC transplantation via the tail vein (CCl₄ (8 W)+HUMSCs (tail v.) group), in the 4th week after the stem cells were transplanted, i.e. the 8th week that CCl₄ was fed, the serum GOT level showed no statistical difference (p>0.05) from that in the 4th week. Comparing this to the 8th week of the CCl₄ (8 W) group, no distinct changes were found (p>0.05) (FIG. 1C). For the rats of CCl₄ (8 W)+HUMSCs (liver) group, in the 4th week after the stem cells were transplanted, i.e. the 8th week that CCl₄ was fed, the value of serum GOT level showed a significant decrease (p<0.05) from that in the 4th week. Comparing the serum GOT level of the CCl₄ (8 W)+HUMSCs (liver) group with that of the normal group, there was no statistical difference (p<0.05) (FIG. 1C).

The serum GPT level of the normal group was 38.8±4.64-48.60±2.97 Unit/L (FIG. 1B). After feeding the rats with CCl₄ for 4 weeks, the serum GPT level increased significantly to 1536.67±305.89 Unit/L (p<0.05). Until the 8th week, the GPT value further increased to 3017.78±562.30 Unit/L (p<0.05) (FIG. 1B). For the rats which received HUMSC transplantation via the tail vein (CCl₄ (8 W)+HUMSCs (tail v.) group), in the 4th week after the stem cells were transplanted, i.e. the 8th week that CCl₄ was fed, the serum GPT level showed no statistical difference (p<0.05) from that in the 4th week. There was also no significant change (p>0.05) (FIG. 1B) in comparing the 8th week of the CCl₄ (8 W) group. For the rats of the CCl₄ (8 W)+HUMSCs (liver) group, in the 4th week after the stem cells were transplanted, i.e. the 8th week that CCl₄ was fed, the serum GPT level decreased significantly (p<0.05) compared to that in the 4th week. Comparing the 8th week of the CCl₄ (8 W) group, there was a significant drop (p<0.05). Comparing the serum GPT level of the normal group, there was no statistical difference (p>0.05) (FIG. 1B).

### Example 2

### Suppression of Liver Fibrosis after HUMSCs Transplantation

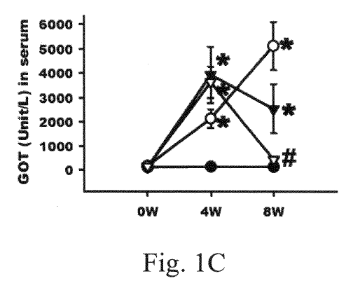

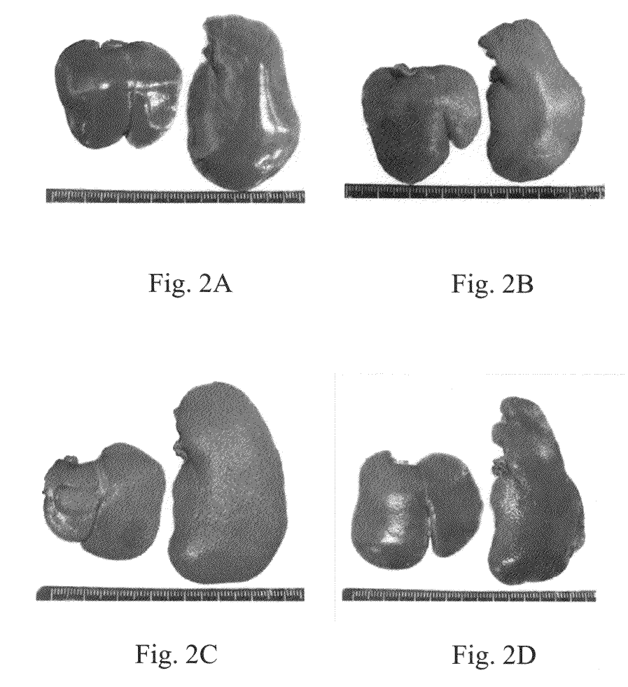

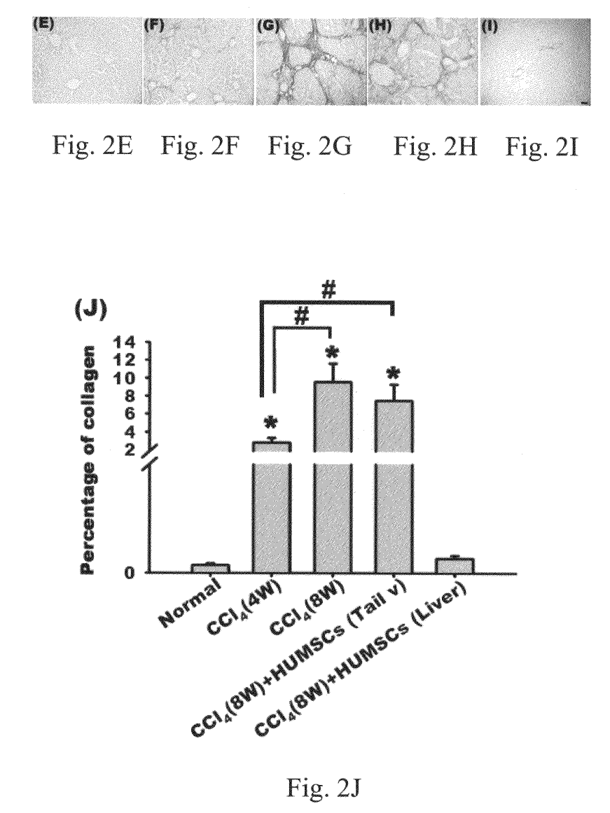

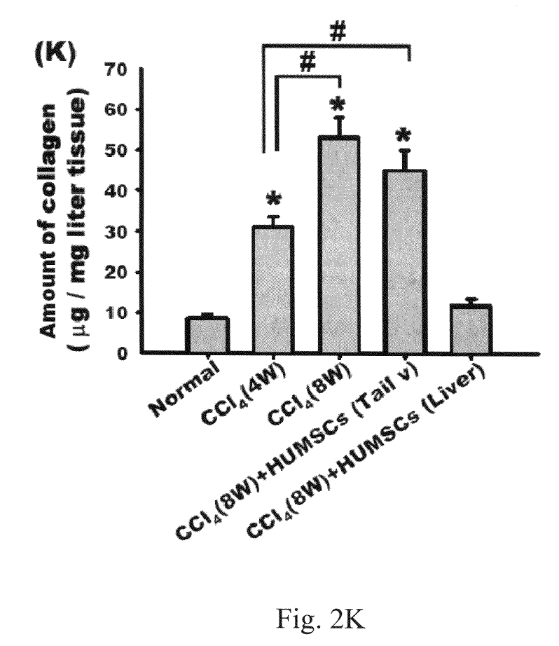

As the livers were observed, it was found that the livers of the normal group were smooth, lustrous, and reddish (FIG. 2A). Regarding the rats received CCl₄ for 4 weeks (CCl₄ (4 W)), the liver surfaces were coarse and relatively bloodless (FIG. 2B). The liver surfaces of the rats fed with CCl₄ for 8 weeks were coarser, with nodules found, and nearly bloodless (FIG. 2C). For the rats of CCl₄ (8 W)+HUMSCs (liver) group, the liver surfaces were slightly coarse, but were more reddish and lustrous compared to those of the CCl₄ (4 W) and CCl₄ (8 W) groups (FIG. 2D).

To determine the changes in liver fibrosis after the rats were fed with CCl₄ and received HUMSC transplantation, sirius red was used to identify the collagen in the liver tissue slices.

The results showed that the expression level of collagen fiber in the liver tissues of the normal group was extremely low (FIG. 2E). The contents of collagen fiber in the liver tissues of the CCl₄ (4 W) group increased significantly (FIG. 2F). After feeding with CCl₄ for 8 weeks (CCl₄ (8 W)), large amounts of collagen fiber appeared in the connective tissue of the inter lobule space (FIG. 2G). For the rats of the CCl₄ (8 W)+HUMSCs (tail v.) group, 4 weeks after the transplantation, the large amount of collagen fiber in the liver tissue still existed (FIG. 2H). For the rats of the CCl₄ (8 W)+HUMSCs (liver) group, 4 weeks after the transplantation, the collagen fiber in the liver tissue improved distinctly (FIG. 2I).

The sirius red-stained areas in the liver slice of each group, or the percentage of sirius red-stained areas, were quantified (FIG. 2J). The results showed that the collagen fiber areas in the livers of the normal group were about 377.65±78.51 μm², occupying 0.07±0.02% of the total liver area. For the rats fed with CCl₄ for 4 weeks, the fibrosis area increased significantly to 11258.89±1716.16 μm , occupying 2.81±0.50% of the total liver area (p<0.05). After feeding the rats with CCl₄ for 8 weeks, the liver fibrosis area increased more significantly to 39377.04±7704.57 μm², occupying 9.56±2.03% of the liver area (p<0.05). For the rats of the CCl₄ (8 W)+HUMSCs (tail v) group, the liver fibrosis area and percentage were approximate to that of the rats fed with CCl₄ for 8 weeks (p>0.05), while there was a statistical increase compared to the rats fed with CCl₄ for 4 weeks (p<0.05). The fibrosis values of the rats of the normal, CCl₄ (4 W), and CCl₄ (8 W) groups were more centralized. However, among the 8 rats in the (8 W)+HUMSCs (tail v) group, the fibrosis value of 6 rats was higher, while that of the other two was lower, which was less centrally distributed. For the rats of the CCl₄ (8 W)+HUMSCs (liver) group, the liver fibrosis area and percentage were approximate to those of the normal group (p>0.05), while there was a statistical drop (p<0.05) compared to those of the CCl₄ (4 W), CCl₄ (8 W), and CCl₄ (8 W)+HUMSCs (tail v) groups.

The collagen content of fresh liver also was quantified (FIG. 2K). The results showed that the collagen level of the normal group was 8.54±1.03 μg collagen per milligram of liver. For the rats of the CCl₄ (4 W) group, the collagen content increased significantly to 30.98±2.76 μg/mg tissue (p<0.05). Feeding the rats until 8 weeks (CCl₄ (8 W)), the collagen level increased more significantly to 53.16±4.83 μg/mg tissue (p<0.05). The collagen level of the CCl₄ (8 W)+HUMSCs (tail v.) group was 45.06±6.02 μg/mg tissue. Comparing the CCl₄ (4 W) and normal groups, there was a statistical increase (p<0.05) (FIG. 2K). For the rats of the CCl₄ (8 W)+HUMSCs (liver) group, the collagen level of the rat livers dropped to reach an approximate level as the normal group (p>0.05). Comparing the CCl₄ (4 W), CC14 (8 W), and CCl₄ (8 W)+HUMSCs (tail v) groups, there was a statistical drop (p<0.05) (FIG. 2K).

GPT and GOP are two well known cytoplasmic hepatocellular enzymes, whose increase in blood is highly indicative for liver damage, e.g. by hepatitis, cirrhosis or hepatic tumors. As shown in the experiment, when the liver was seriously damaged by CCl₄, the level of GPT and GOP went up dramatically, as did the collagen level. However, both groups of rats having tail injection or liver injection with HUMSCs significantly showed relief from the liver damage, wherein the GPT, GOP, and percentage of collagen are low, and the surface of the liver is more reddish and lustrous. This indicates that HUMSCs effectively suppress the progress of liver fibrosis, when the liver suffers continuous damage.

### Example 3

### Inhibition of Liver Inflammation and Enhancement of Liver Regeneration after HUMSCs Transplantation

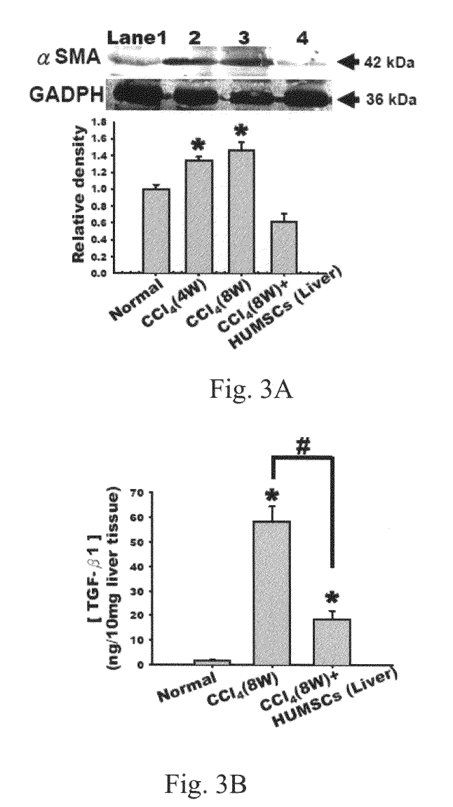

Western blotting was used to quantify the α-smooth muscle actin (SMA) level in the livers. The results showed that the α-SMA levels of groups (CCl₄ (4 W)) and (CCl₄ (8 W)) increased compared to that of the normal group at 135%-147% (p<0.05). The α-SMA level in the rats of the CCl₄ (8 W)+HUMSCs (liver) group approximated the level of the normal group (p>0.05). Comparing the CCl₄ (4 W) and CCl₄ (8 W) groups, there was a statistical decrease (p<0.05) (FIG. 3A).

The TGF-β1 levels in the fresh livers were also quantified. The results showed that the TGFβ1 level of the CCl₄ (8 W) group increased significantly (p<0.05) compared to that of the normal group. The TGF-β1 level of the CCl₄ (8 W)+HUMSCs (liver) group was less than that of the CCl₄ (8 W) group (p<0.05); compared to that of the normal group, there was a significant increase (p<0.05) (FIG. 3B).

Western blotting was then used to quantify the Met-P level in the livers. The results showed that the Met-P concentrations of the CCl₄ (4 W) and CCl₄ (8 W) groups were approximate to that of the normal group (p>0.05) (FIG. 3C). For the rats of the CCl₄ (8 W)+HUMSCs (liver) group, there was a statistical increase in Met-P level compared to those of the normal, CCl₄ (4 W), and CCl₄ (8 W) groups (p<0.05) (FIG. 3C).

Real time RT-PCR was used to quantify the relative contents of rat hepatic growth factor (HGF) in fresh livers. The results showed that there was no statistical difference (p>0.05) between the rat HGF levels of the CCl₄ (8 W) group and the normal group. The rat HGF level of the CCl₄ (8 W)+HUMSCs (liver) group increased significantly compared to those of the normal group and the CCl₄ (8 W) group (p<0.05) (FIG. 3D).

It was confirmed by these results that, in vivo, the expression of α-SMA and TGF-β1 was a reliable marker of hepatic satellite cells activation which preceded fibrous tissue deposition. HUMSCs inhibited the expression of α-SMA and TGF-β1 and prevented the liver from fibrosis and inflammation. Moreover, the results also showed that HUMSCs stimulated the expression of HGF and activated its receptors, Met-P, to induce an effect of wound healing at the damaged site of the liver.

### Example 4

### Fate of HUMSCs Existed in the Grafted Livers

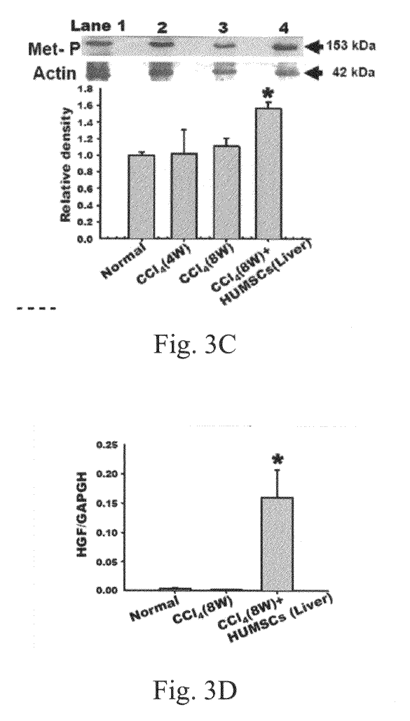

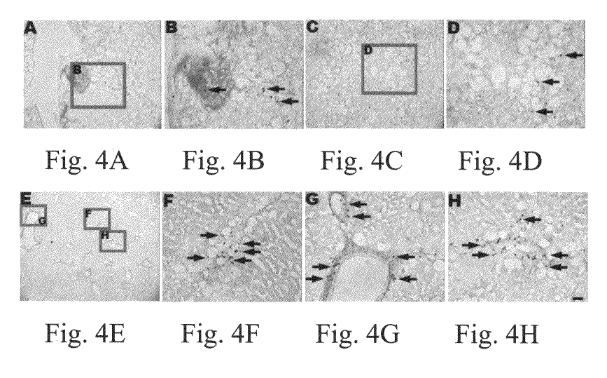

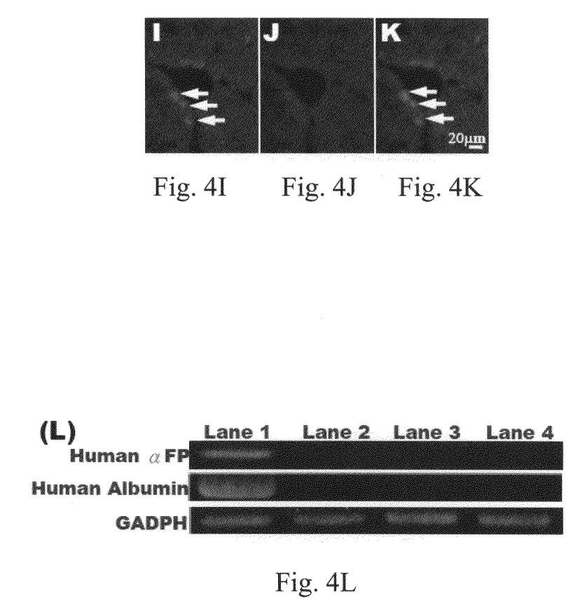

The liver tissue slices of the CCl₄ (8 W)+HUMSCs (tail v) group were immunostained by anti-human specific nuclei antigen in order to trace the existence of HUMSCs. It was found that HUMSCs existed in rat livers 4 weeks after HUMSCs were transplanted, but the amount was not much (FIGS. 4A, B, C and D).

As shown in the lower magnification of the liver tissue slices, the fibrosis condition of the CCl₄ (8 W)+HUMSCs (liver) group was reduced and was restricted to around the central vein, portal triad and interlobule connective tissue after HUMSC transplantation. Large amounts of HUMSCs also existed in these areas (FIGS. 4E, F, G and H).

In order to identify HUMSC differentiation into hepatocytes, double immunostaining of anti-human specific nuclei antigen and anti-human albumin were performed. The results showed that HUMSCs were not found in the rat livers of the normal group; neither was human albumin positive cell distribution (data not shown). The liver sections of the CCl₄ (8 W)+HUMSCs (liver) group were stained by double immunohistochemical staining for anti-human specific nuclear antigen and human albumin, and showed a complete absence of human albumin (FIGS. 4I-K).

RT-PCR was used to verify the existence of human a fetoprotein and human albumin in the rat livers. Human a fetoprotein and human albumin were not identified in the grafted rat livers of the CCl₄ (8 W)+HUMSCs (liver) group (FIG. 4L).

The stained tissue studies indicate that HUMSCs, which are directly injected to the liver or systemically injected into the tail vein, will stay or migrate to the liver damaged site. The HUMSCs also effectively reduce and limit the fibrosis around the central vein, portal triad and interlobule connective tissue, as shown in the liver slices.

### Example 5

### Expression level of Human CTACK, LIF, and Prolactin in the Fibrous Liver after HUMSC Transplantation

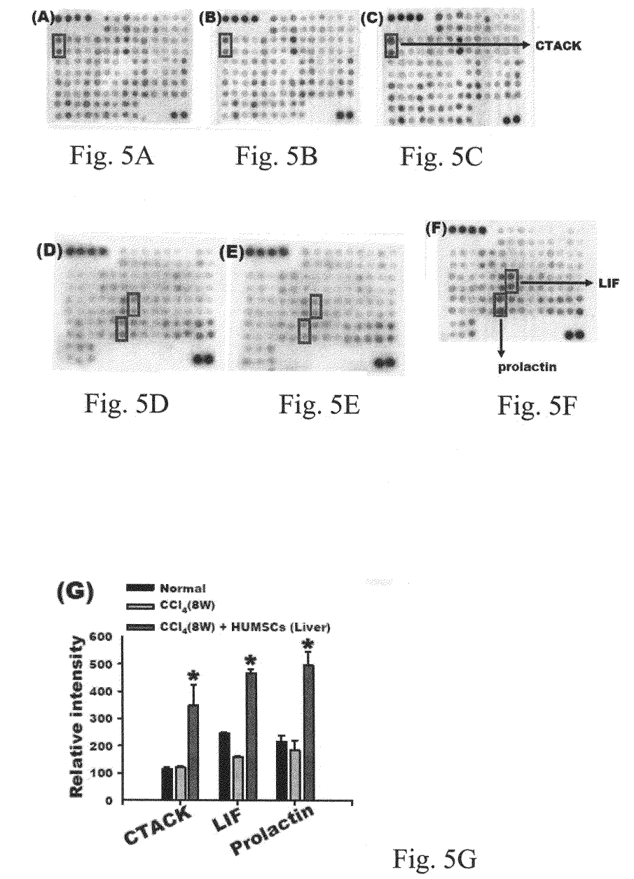

Liver protein from the rats of the normal, CCl₄ (8 W), and CCl₄ (8 W)+HUMSCs (liver) groups were prepared and incubated with membranes containing an array of 174 human protein cytokine antibodies. Autoradiographs were scanned, and the density of each cytokine at the corresponding position was determined. The relative intensities of each cytokine were normalized to control spots on the same membrane. Major increases among cytokines are represented graphically in FIGS. 5A-F. Human CTACK, LIF, and prolactin were significantly increased in the rat livers of the CCl₄ (8 W)+HUMSCs (liver) group (p<0.05) (FIG. 5G).

CTACK and LIF are both well know mitogen for stimulating the growth of stem cells. Therefore, according to the result shown in Example 5, HUMSCs induced the production of CTACK and LIF in the liver, and these mitogen further stimulated the growth of HUMSCs and liver stem cell to replace death liver cells after chemicals damage.

### Example 6

### Transplantation of HUMSCs to Fibrosis Livers Decreased Liver Inflammation

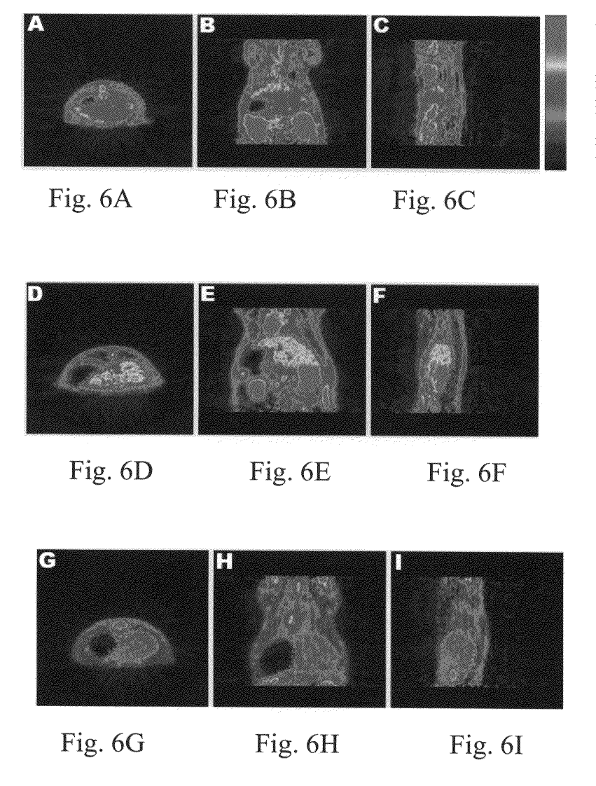

Four weeks after transplantation of 5×10⁵ HUMSCs into the fibrosis rat livers, ¹⁸F-FDG was injected into the rats and microPET™ was used to detect hepatic inflammation or track the existence of hepatoma. It was found that metabolism of the liver was extremely low before CCl₄ treatment (FIGS. 6A-C). After CCl₄ application for 4 weeks, the metabolism or inflammatory levels of the rat livers became higher (FIGS. 6D-F). Four weeks after transplantation of HUMSCs, the metabolism level in the grafted livers decreased and reverted to the level of pre-treatment, indicating the inflammatory response was decreased, and hepatoma existence was undetected (FIG. 6G-I).

It was provide by these results that HUMSCs decreased liver inflammation in Example 3. In summary, HUMSCs had an ability to treat a liver disease or aided recovery from a liver disease by inducing HGF expression and inhibiting the production of inflammation factors in the liver, such as α-SMA and TGF-β1 expression.

All publications cited herein are incorporated herein by reference.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A method for treating a liver disease or aiding recovery from a liver disease in a subject comprising transplanting human umbilical mesenchymal stem cells (HUMSCs) obtained from Wharton's Jelly to areas of the liver disease of the subject.

2. The method according to claim 1, wherein the transplantation of HUMSCs is achieved by direct injection of HUMSCs to the areas of the liver disease.

3. The method according to claim 1, wherein the liver disease is selected from the group consisting of liver inflammation, liver steatosis, liver fibrosis, liver cirrhosis, and hepatitis.

4. The method according to claim 3, wherein the liver fibrosis is the result of chronic injury.

5. A method for enhancing liver regeneration and inhibiting liver inflammation in a subject with liver disease comprising transplanting human umbilical mesenchymal stem cells (HUMSCs) obtained from Wharton's Jelly to areas of liver disease of the subject.

6. The method according to claim 5, wherein the liver disease is selected from the group consisting of liver inflammation, liver steatosis, liver fibrosis, liver cirrhosis, and hepatitis.

7. The method according to claim 5, wherein the transplantation of HUMSCs is achieved by direct injection of HUMSCs to the areas of liver disease.

8. The method of claim 5, wherein the liver disease is liver fibrosis.

9. The method according to claim 8, wherein the liver fibrosis is the result of chronic injury.
